(19) United States
(12) Patent Application Publication
(10) Pub. No.: US 2024/0398999 A1
(43) Pub. Date: Dec. 5, 2024

(54) DC BIOMIMETIC MEMBRANE NANOPARTICLE LOADED WITH NIR-II AIE DYE, PREPARATION METHOD THEREFOR AND USE THEREOF

(71) Applicants: SHENZHEN INSTITUTES OF ADVANCED TECHNOLOGY CHINESE ACADEMY OF SCIENCES, Shenzhen (CN); THE HONG KONG UNIVERSITY OF SCIENCE AND TECHNOLOGY, Hong Kong (CN)

(72) Inventors: Lintao CAI, Shenzhen (CN); Xing YANG, Shenzhen (CN); Ping GONG, Shenzhen (CN); Pengfei ZHANG, Shenzhen (CN); Benzhong TANG, Hong Kong (CN); Tsz Kin KWOK, Hong Kong (CN); Ting YANG, Shenzhen (CN); Qiqi LIU, Shenzhen (CN); Xiuwen ZHANG, Shenzhen (CN); Xinghua YU, Shenzhen (CN); Luo HAI, Shenzhen (CN)

(73) Assignees: SHENZHEN INSTITUTES OF ADVANCED TECHNOLOGY CHINESE ACADEMY OF SCIENCES, Shenzhen (CN); THE HONG KONG UNIVERSITY OF SCIENCE AND TECHNOLOGY, Hong Kong (CN)

(21) Appl. No.: 18/326,899

(22) Filed: May 31, 2023

(51) Int. Cl. A61K 49/00; A61K 41/00; A61P 35/00

(52) U.S. Cl. CPC A61K 49/0093; A61K 41/0052; A61K 49/0021; A61P 35/00

(57) ABSTRACT

The present invention belongs to the field of nanomedicine, and provides a DC biomimetic membrane nanoparticle loaded with an NIR-II AIE dye, a preparation method therefor and use thereof. The DC biomimetic membrane nanoparticle loaded with the NIR-II AIE dye comprises a DC membrane and an NIR-II AIE dye loaded therein, wherein the NIR-II AIE dye has photothermal characteristics. The DC biomimetic membrane nanoparticle loaded with the NIR-II AIE dye provided by the present invention not only solves the problem that the NIR-II AIE dye is poor in water solubility and limited in fluorescence imaging, but also increases the enrichment efficiency of the NIR-II AIE dye coated by the DC membrane at a tumor site and successfully activates the activity of T cells in vivo.

### TECHNICAL FIELD

The present invention belongs to the field of nanomedicine, and particularly relates to a DC biomimetic membrane nanoparticle loaded with an NIR-II AIE dye, a preparation method therefor and use thereof.

### BACKGROUND

Near-infrared (NIR) light has been widely used for photothermal therapy due to its superior tissue penetration, remote controllability, and high-resolution adjustability in time and space. Compared with the near-infrared one-zone (NIR-I, 700-900 nm) fluorescence imaging, the near-infrared two-zone (NIR-II, 900-1700 nm) fluorescence imaging has the advantages of lower autofluorescence, higher signal-to-noise ratio, larger penetration depth and the like. Therefore, NIR-II is widely used as an emerging field of research for in vivo imaging and for image-guided photothermal therapy.

Most of the traditional NIR-II organic fluorescent dyes can generate aggregation fluorescence quenching (ACQ), and the phenomenon of fluorescence intensity reduction caused by intermolecular π-π accumulation influences the optical properties of the dyes, thereby restricting the application prospects of the dyes. To solve the problems caused by ACQ of traditional dyes, the team led by academician Benzhong Tang proposed in 2001 the concept of molecular aggregation-induced emission (AIE), which was based on the principle of intramolecular motion limitation. The AIE dye has a stronger emission in a focused state than the conventional ACQ organic dye. Near-infrared two-zone AIE dyes overcome limitations in penetration depth and fluorescence efficiency, providing high-performance fluorescence imaging with greater accuracy. Based on the advantages of near-infrared two-zone AIE molecules, they have been used for biofluorescence imaging of blood vessels, bile ducts, and the gastrointestinal tract.

The majority of the NIR-II AIE fluorescent dyes are not water soluble, thus limiting their use.

### SUMMARY

In order to solve the problems of the prior art that the NIR-II AIE fluorescent dye is insoluble in water and has limited application in fluorescence imaging and the problem of reduced thermal sensitivity of tumor cells, the present invention provides a dendritic cell (DC) biomimetic membrane nanoparticle loaded with an NIR-II AIE dye, which comprises a DC membrane and an NIR-II AIE dye loaded thereon, wherein the NIR-II AIE dye has photothermal characteristics.

Further, the DC membrane and the NIR-II AIE dye loaded thereon are subjected to the following method to obtain the DC biomimetic membrane nanoparticle loaded with the NIR-II AIE dye: dispersing DC cell membranes in PBS solution, then injecting the solution of the DC cell membranes through a filter by using a syringe, repeatedly operating so as to enable the cell membranes to be adhered to a filter membrane, aspirating the NIR-II AIE dye by using the syringe, enabling the NIR-II AIE dye to pass through the filter membrane having the cell membranes adsorbed thereto, and performing extrusion and filtration until the DC biomimetic membrane nanoparticle loaded with the NIR-II AIE dye is obtained.

Further, the NIR-II AIE dye is a BPBBT dot.

Further, the BPBBT dot is prepared by the following method: dissolving DSPE-PEG-2000 and BPBBT in an organic solvent, dropwise adding the mixed solution into deionized water under an ultrasonic condition, continuously carrying out ultrasonic treatment until the mixed solution forms a uniform emulsion, completely blowing the organic solvent in the aqueous solution by an inert gas, and filtering the obtained aqueous solution by a filter to obtain the BPBBT dot.

The present invention also provides a method for preparing a DC biomimetic membrane nanoparticle loaded with an NIR-II AIE dye.

The method for preparing the DC biomimetic membrane nanoparticle loaded with the NIR-II AIE dye comprises the following steps:

S1: preparing the NIR-II AIE dye; and

S2: dispersing DC cell membranes in PBS solution, then injecting the obtained solution of the DC cell membranes through a filter by using a syringe, repeatedly operating so as to enable the cell membranes to be adhered to a filter membrane, aspirating the NIR-II AIE dye prepared in S1 by using the syringe, enabling the NIR-II AIE dye to pass through the filter membrane having the cell membranes adsorbed thereto, and performing extrusion and filtration until the DC biomimetic membrane nanoparticle loaded with the NIR-II AIE dye is obtained.

Further, the NIR-II AIE dye in S1 is a BPBBT dot and is prepared by the following method:
dissolving DSPE-PEG-2000 and BPBBT in an organic solvent, dropwise adding the mixed solution into deionized water under an ultrasonic condition, continuously carrying out ultrasonic treatment until a uniform emulsion is formed, removing the organic solvent in the emulsion, and filtering the obtained emulsion by using a filter to obtain the BPBBT dot.

Further, a mass ratio of DSPE-PEG-2000 to BPBBT in S1 is 5:1 to 5:5, preferably 5:1, 5:2, 5:3, 5:4, or 5:5.

Further, concentrations of DSPE-PEG-2000 and BPBBT in S1 in the organic solvent are 5 mg/mL and 1-5 mg/mL, respectively.

Further, the ultrasonic treatment in S1 is carried out at a power of 100 W and a frequency of 40 KHz.

Further, the method for obtaining the DC cell membrane in S2 comprises: isolating immature DCs from mouse bone marrow; stimulating with 4T1 cells and Poly I:C to obtain mature DCs, and then extracting to obtain DC cell membranes.

Further, the filter membrane for the filter in S2 is a 0.22 μm filter membrane.

The present invention also provides use of the DC biomimetic membrane nanoparticle loaded with the NIR-II AIE dye according to any one of the above in the preparation of a reagent for in vivo imaging, a reagent for image-guided photothermal therapy and a reagent for drug delivery systems.

Further, the in vivo imaging or imaging guidance is in vivo imaging or imaging guidance in blood vessels, bile ducts, and gastrointestinal tracts in vivo.

The present invention also provides use of the DC biomimetic membrane nanoparticle loaded with the NIR-II AIE dye according to any one of the above in the preparation of a photothermal therapeutic drug or a photothermal therapeutic sensitizer for the treatment of a tumor.

The present invention also provides use of the DC biomimetic membrane nanoparticle loaded with the NIR-II AIE dye according to any one of the above in the preparation of a drug or reagent for inhibiting expression of HSP70 at tumor sites, increasing thermal sensitivity of tumor cells and activating activity of T cells in vivo.

The present invention also provides a method for inhibiting a tumor comprising administering to a subject the DC biomimetic membrane nanoparticle loaded with the NIR-II AIE dye according to any one of the above.

Further, the method also comprises further performing low-temperature photothermal therapy.

Further, the tumor includes lung cancer, melanoma, breast cancer, prostate cancer, colon cancer, renal cell carcinoma, ovarian cancer, pancreatic cancer, hepatocellular carcinoma, and rhabdomyosarcoma.

The present invention also provides a method for detecting a tumor comprising administering to a subject the DC biomimetic membrane nanoparticle loaded with the NIR-II AIE dye according to any one of the above, and imaging the AIE dye therein.

The present invention also provides a photothermal therapeutic drug or reagent comprising the DC biomimetic membrane nanoparticle loaded with the NIR-II AIE dye according to any one of the above.

### Beneficial Effects

In the intelligent nanoparticle-DC@BPBBT dot coated by the DC membrane provided by the present invention, the BPBBT dot has the capability of NIR-II fluorescence imaging and photothermal conversion, and the outer DC membrane retains the capability of antigen presentation and T cell loading. The problem that the water solubility of the NIR-II AIE dye is poor and limited in fluorescence imaging is solved, meanwhile, the enrichment efficiency of the NIR-II AIE dye coated by the DC membrane in a tumor site is improved, and the activity of T cells in vivo is successfully activated, so that the tumor necrosis factor (TNF-α) is secreted to inhibit the expression of HSP70 in the tumor site, the thermal sensitivity of the tumor cells is increased, the growth of the tumor is inhibited under low-temperature photothermal therapy, and the diagnosis and treatment integration of the tumor is achieved.

### BRIEF DESCRIPTION OF DRAWINGS

FIG. 1 is a flow chart of the preparation of the biomimetic nanoparticle DC@BPBBT dot provided by the present invention;

FIG. 2 is a characterization diagram of BPBBT, BPBBT dots and DC@BPBBT dots provided by the present invention;

FIG. 3 is a characterization diagram of particle size and morphology of BPBBT dots and DC@BPBBT dots provided by the present invention:

FIG. 4 shows the potential difference between BPBBT dot and DC@BPBBT dot by SDS-PAGE determination:

FIG. 5 shows the results of staining of SDS-PAGE gel confirming the DC a, BPBBT dot by SDS-PAGE determination in Effect Example 4:

FIG. 6 shows the fluorescence intensity of DC@BPBBT dot solution at 980 nm with time;

FIG. 7 shows the in vitro results of good photothermal stability of BPBBT:

FIG. 8 shows the distribution of BPBBT dot or DC@BPBBT dot in mice after intravenous injection in Effect Example 7:

FIG. 9 shows the results of the cytotoxicity evaluation:

FIG. 10 shows the results of in vivo biocompatibility and toxicity evaluations:

FIG. 11 shows the killing efficiency of DC@BPBBT dot cells in vitro in Effect Example 10:

FIG. 12 shows the results of T cell injury by DC@BPBBT dots:

FIG. 13 shows the results of the amount of TNF-α after treatment with DC@BPBBT dot:

FIG. 14 shows the expression results of HSP70 protein in 4T1 cancer cells by evaluation using western blot:

FIG. 15 shows the effect of DC@BPBBT dot on in vivo tumor treatment:

FIG. 16 shows photoluminescence (PL) spectra of HSP70 protein at different groups of tumor sites:

FIG. 17 shows the results of the detection of the expression of HSP70 protein by western blot using beta-actin as an internal reference after different treatments:

FIG. 18 shows representative flow cytometry plots of activated T cells in the spleens of 4 groups:

FIG. 19 is a schematic of in vivo tumor imaging guidance and photothermal therapy at mild temperatures.

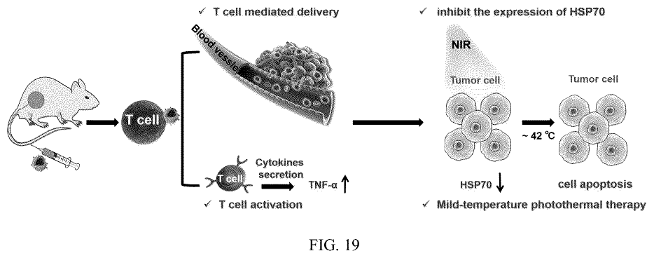

### DETAILED DESCRIPTION OF THE EMBODIMENTS

In order to make the aforementioned objects, features and advantages of the present invention more comprehensible, embodiments accompanied with figures are described in detail below; but are not to be construed as limiting the implementable range of the present invention.

In order to improve the problem that most of the NIR-II AIE fluorescent dyes are not water-soluble, nanotechnology provides a method. Nanotechnology also plays a crucial role in improving the efficiency of loading and circulation time of these molecules in vivo, especially in tumor-targeted drug delivery systems and imaging. Currently, common modifications include: enzymes, antibodies, aptamers, small molecules, and other functional groups with targeting properties, they are loaded onto the surface of the nanoparticle through various chemical or non-chemical interactions, and these molecules help the nanoparticle to recognize receptors specifically expressed in tumor cells or tumor microenvironment. Despite the excellent therapeutic effects of the nanoparticle, there are still deficiencies in multiple functions of circulation enhancement, targeting specificity, immunomodulation and immune homing.

In recent years, cell-mediated drug delivery systems have become an effective strategy to address the challenges faced by the nanoparticle. This novel approach takes advantage of the long circulation of cells in the blood, abundant surface ligands (facilitating intercellular specificity loading), tumor targeting, and flexible cell morphology, challenge biological barriers, and maximize therapeutic efficacy and minimize associated side effects by cell-to-cell signaling. Currently, cell membrane-coated nanoparticles are of interest because they mimic the characteristics of the cell surface, which not only help reduce the immune rejection of the nanoparticle by the body, but also effectively combine the properties of natural and synthetic materials. The drug carrier based on cells and cell membranes is used, so that immune escape is increased, tumor targeting is enhanced, and enrichment of the nanomaterial at a tumor site is further enhanced. Furthermore, an expected immune regulation effect can be generated through cell surface engineering, so that growth and deterioration of tumors are inhibited. The present invention aims to achieve fluorescence imaging and delivery of the nanoparticle loaded with the NIR-II AIE dye through the DC biomimetic membrane by utilizing the characteristics of the biomimetic membrane and the nanoparticle, thereby achieving diagnosis and treatment integration of tumors.

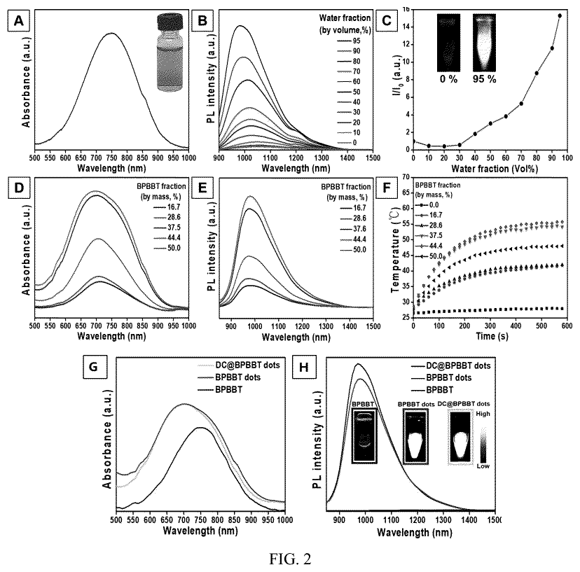

BPBBT (CAS number: 1070910-81-4 formula: C₇₄H₉₂N₆O₄S₂) is a lipophilic donor-acceptor-donor (D-A-D) fluorophore with maximum fluorescence emission and AIE effect in NIR-II. The absorption properties of BPBBT were investigated using ultraviolet-visible spectroscopy. As shown in FIG. 2A, BPBBT has a broad absorption band in tetrahydrofuran (THF) of 500-1000 nm, with one peak at about 750 nm. The AIE properties of BPBBT were studied by monitoring the fluorescence fluctuations of the tetrahydrofuran/water mixture using photoluminescence (PL) spectroscopy (FIGS. 2B and 2C). In pure polar THF solvent, BPBBT is not fluorescent, as the fluorescence quenching effect of BPBBT depends on the polarity of the solvent. In the mixture of THF and water, the fluorescence of BPBBT reappeared and gradually increased. When the volume fraction of the tetrahydrofuran water is 0%-30%, the polarity of the solvent is further improved and the fluorescence intensity of BPBBT is reduced due to the distorted state of charge transfer in molecules. Whereas the fluorescence intensity of BPBBT increases with increasing water fraction in the mixture, with a maximum fluorescence intensity of 95%. Further, due to the AIE state, when the water fraction is 80% or more, the emission peak undergoes a blue shift. This phenomenon is attributed to the formation of nanodots of BPBBT in pure polar solvents, such as mixtures of large proportions of water (>80%), limiting intramolecular rotation, resulting in a blue-shift of the absorption and emission spectra compared to the spectra of BPBBT in THF. In the NIR-II window; the emission tail of BPBBT extends above 1000 nm, indicating that BPBBT is an NIR-II AIE active molecule.

The present invention takes BPBBT as an example to explain the DC biomimetic membrane nanoparticle loaded with the NIR-II AIE dye, the preparation method therefor and the use thereof.

Material: Analytical grade CHCl₃ was purchased from China Lingfeng Chemical Reagent Co., Ltd. 1,2-distearoyl-sn-glycerol-3-phosphoethanolamine-n-[amino (polyethylene glycol)2000] (DSPE-PEG-2000) was obtained from Postobio Inc. (China). BPBBT, 4,8-bis[4-(N, N-bis(4-octyloxyphenyl)amino)phenyl]benzo[1,2-c:4,5-c′]bis([1,2,5]thiadiazole) was synthesized by Wuxi Advanced Materials, Inc. mIL-4, mGM-CSF and mIL-2 were purchased from Biotech Co., Ltd. The CCK-8 kit and Calcein/PI cell viability/cytotoxicity assay kit were purchased from Beyotime Biotech Co., Ltd. RPMI 1640, Dulbecco's Modified Eagle Medium (DMEM), penicillin-streptomycin and fetal bovine serum were all from Gibco.

Instrument: absorption spectra and photoluminescence spectra were measured on a PerkinElmer Lambda 25 ultraviolet-visible absorption spectrophotometer and an Edinburgh F900 fluorescence spectrometer equipped with a xenon arc lamp, respectively. The surface charge and hydrodynamic size of the nanoparticle were measured at 25° C. using a zeta-size-Nano ZS (Malvern, UK) instrument. Transmission electron microscopy (TEM) images were recorded using Tecnai G2 F20 S-TWIN (FEI). TEM samples were prepared by dropping the nanoparticle solution onto a 200 mesh copper screen and then drying the sample in a clean window at 25° C. Flow cytometry (BECKMAN COULTER) was used for flow cytometry analysis. And detection of the protein content of the DC membrane is by an SDS-PAGE gel electrophoresis method. 808 nm laser (Lasever Inc., China) was used as in vivo and in vitro treatment light source.

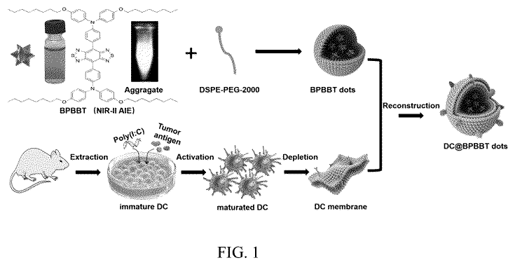

Referring to the drawings of the specification, FIG. 1, the method for preparing the nanoparticle loaded with the NIR-II AIE dye by coating NIR-IIAIE-BPBBT nanoparticle with a DC membrane comprises the following steps.

Synthesizing a BPBBT dot: dissolving DSPE-PEG-2000 and BPBBT in an organic solvent, then dropwise adding the mixed solution into deionized water under an ultrasonic condition, and then continuously carrying out ultrasonic treatment to obtain a mixed solution to form a uniform emulsion: blowing off the organic solvent in the aqueous solution by using an inert gas, filtering the obtained aqueous solution multiple times by using a filter to obtain a clear BPBBT dot solution, and storing the clear BPBBT dot solution in a refrigerator at 4° C. for later use:

in order to dissolve the BPBBT molecule in vivo, DSPE-PEG-2000 is used as a nanocarrier material because DSPE-PEG-2000 has biocompatibility, biodegradability and amphiphilicity, and can be used to prolong blood circulation time, improve stability and improve encapsulation efficiency.

\textcircled{2} Synthesis of DC@BPBBT dot: resuspending the extracted DCs cell membrane in PBS solution, placing on ice, fully resuspending the cell membranes by using an ultrasonic cell disruption instrument, then injecting the obtained resuspended cell membrane solution through a filter by using a syringe, and repeating the operation for at least 10 times, so that the cell membranes are fully adhered to the filter membrane: then, the BPBBT dot was passed through a filter having a cell membrane adsorbed thereto by a syringe, and the resulting solution was subjected to multiple extrusion and filtration to obtain a DC@BPBBT dot solution, which was stored in a refrigerator at 4° C. for later use.

Example 1 is a method of a nanoparticle loaded with an NIR-II AIE dye comprising the steps of:

(1) Synthesis of BPBBT dot. 5 mg of DSPE-PEG-2000 and 4 mg of BPBBT were dissolved in 1 mL of CHCl₃, and the mixed solution was added dropwise to 5 mL of deionized water under an ultrasonic condition, and then ultrasonic treatment was continuously carried out at a frequency of 40 kHz and a power of 100 W for 10 to 20 min. The aqueous solution was then purged of CHCl₃ with nitrogen and the resulting aqueous solution was filtered through a 0.22 μm filter several times to give a clear BPBBT dot solution which was stored in a refrigerator at 4° C. until use.

(2) Synthesis of DC a, BPBBT dot. Immature DCs were isolated from mouse bone marrow; and after stimulation with 4T1 cells and Poly I:C, mature DCs were obtained, and then cell membranes were extracted. The extracted DC cell membranes were resuspended in PBS solution and placed on ice, the cell membranes were fully resuspended using a sonicator, and then the resulting resuspended cell membrane solution was injected through a 0.22 μm filter using a syringe, and the operation was repeated at least 10 times, thereby allowing the cell membranes to sufficiently adhere to the filter membrane. then, the BPBBT dot was passed through a filter having a cell membrane adsorbed thereto by a syringe, and the resulting solution was subjected to multiple extrusion and filtration to obtain a DC a, BPBBT dot solution, which was stored in a refrigerator at 4° C. for later use.

### Example 2

Example 2 is substantially the same as Example 1, except that (1) 5 mg of DSPE-PEG-2000 and 1 mg of BPBBT were used in the synthesis step of the BPBBT dot.

### Example 3

Example 3 is substantially the same as Example 1, except that (1) 5 mg of DSPE-PEG-2000 and 2 mg of BPBBT were used in the synthesis step of the BPBBT dot.

### Example 4

Example 4 is substantially the same as Example 1, except that (1) 5 mg of DSPE-PEG-2000 and 3 mg of BPBBT were used in the synthesis step of the BPBBT dot.

### Example 5

Example 5 is substantially the same as Example 1, except that (1) 5 mg of DSPE-PEG-2000 and 5 mg of BPBBT were used in the synthesis step of the BPBBT dot.

BPBBT, the BPBBT dots prepared in Examples 1-5, or the DC@BPBBT dot, were characterized or tested as follows, wherein the animals and tumor models mentioned in the present invention were treated according to the Guide for the Care and Use of Laboratory Animals. The program is approved by the Animal Protection and Utilization Committee (Shenzhen Institute of Advanced Technology, Chinese Academy of Sciences). Female BALB/C mice aged 6-8 weeks (Vitamin River Laboratory Animal Science Co., Ltd., CHN) were injected with 4T1 cells (1×10⁷) subcutaneously in the abdomen. The tumor volume is calculated as follows: volume=length×width²×0.5.

### Effect Example 1. Characterization of BPBBT, BPBBT Dot and DC@BPBBT Dot

The ultraviolet absorption, photoluminescence spectra and photothermal conversion effects of BPBBT, the BPBBT dot prepared in Example 1, or the DC@BPBBT dot were determined.

A in FIG. 2 is the ultraviolet-visible absorption spectrum of BPBBT in tetrahydrofuran (THF) and the photograph of a solution of BPBBT in THF:

B in FIG. 2 is the photoluminescence (PL) spectrum of BPBBT (10 μM) in a 0%-95% water/THF mixture at 808 nm excitation:

C in FIG. 2 is a graph of I/I₀ of BPBBT as a function of water fraction in a water/THF mixture, I and I₀ show the fluorescence intensity of BPBBT (10 μM) in water/THF mixtures and THE mixtures of varying water contents, respectively, and NIR-II FL of BPBBT at 0% and 95% water fractions;

D in FIG. 2 is the absorption spectrum of BPBBT dots of different mass ratios (BPBBT: DSPE-PEG-2000) in water:

E in FIG. 2 is the PL spectrum of BPBBT dot in water at different mass ratios (BPBBT: DSPE-PEG-2000);

F in FIG. 2 is the temperature-time curve of BPBBT dot in water at different mass ratios (BPBBT: DSPE-PEG-2000) under 808 nm laser irradiation:

G in FIG. 2 is the absorption spectrum of BPBBT dot, DC a, BPBBT dot in water and BPBBT in tetrahydrofuran (THF):

H in FIG. 2 is the photoluminescence spectrum of BPBBT dot, DC@BPBBT dot in water and BPBBT in tetrahydrofuran (THF) and respectively shows NIR-II fluorescence images of BPBBT dot, DC@BPBBT dot and BPBBT in THE solutions.

### Effect Example 2. Characterization of Particle Size and Morphology

The particle size or morphology of the BPBBT dot and DC@BPBBT dot prepared in Example 1 was characterized to produce particles of DC@BPBBT dot having a particle size of 147.2 nm.

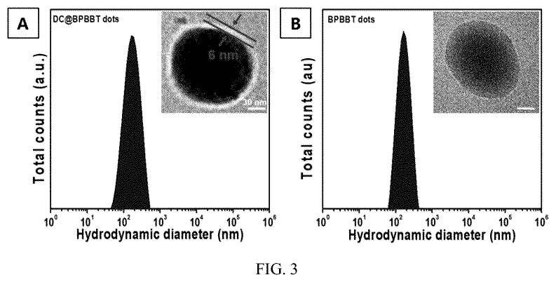

A in FIG. 3 is the DLS hydrodynamic diameter measurement result of the DC/a, BPBBT dot and a TEM image of the DC@BPBBT dot:

B in FIG. 3 is the DLS hydrodynamic diameter measurement result of the BPBBT dot and a TEM image of the BPBBT dot.

DC@BPBBT dot and BPBBT dot hydrodynamic diameter sizes demonstrated that the resulting BPBBT dot and DC@BPBBT dot particles were both nano-scale and uniform in size, confirming that BPBBT dot nanoparticles have been successfully packaged as DC@BPBBT dot particles.

### Effect Example 3. Potential Measurement

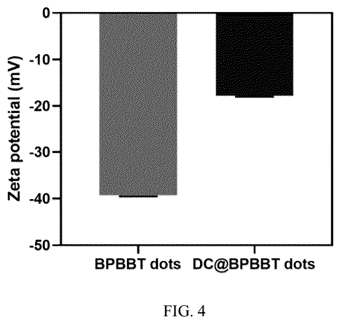

FIG. 4 shows SDS-PAGE measurements of the potential difference between BPBBT dot and DC@BPBBT dot. Before and after the envelope, the potential difference appears, and further proves that the DC cell membrane is successfully coated on the surface of the BPBBT dot particle to form the DC@BPBBT dot particle.

### Effect Example 4. Measurement of Membrane Protein Composition of DC@BPBBT Dot DC

The membrane protein composition of the surface DC of the DC@BPBBT dot is analyzed by SDS-PAGE electrophoresis and compared with the original mature DC membrane protein.

Preparation of cell membrane protein sample: adding the extracted cell membrane components into 5×SDS-PAGE protein loading buffer solution at a ratio of 4:1, diluting the SDS-PAGE protein loading buffer solution in the mixed solution to 1×, and boiling the mixed solution for 10 min at 100° C. by using a metal bath to fully denature and dissolve the proteins.

Detecting the total protein content of the cell membrane: preparing 12% SDS-PAGE gel according to the instruction in the kit for preparing SDS-PAGE gel, calculating the amount of the sample according to the concentration of the total protein detected by a microplate reader, ensuring the same total protein content of the sample, make the gel move by using a small vertical electrophoresis apparatus 80 V, and cutting off the power supply when the sample moves to the bottom of the gel: staining 12% SDS-PAGE gel according to Coomassie brilliant blue fast stain instructions and analyzing total protein content.

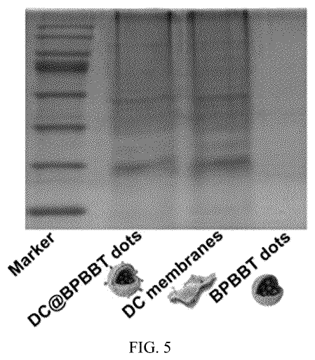

The results in FIG. 5 show that the method of the present invention has successfully coated the DC cell membrane on the surface of the BPBBT dot particle, and meanwhile, the method of the present invention maintains the protein amount of the DC membrane surface, which proves that the DC membrane surface characteristics are not changed and still have the mature DC cell membrane characteristics.

### Effect Example 4. Measurement of Photothermal Conversion Efficiency of BPBBT Dot

Under the irradiation of an 808 nm laser, the BPBBT dot prepared in Example 1 at a concentration of 50 μM was heated for 3 min and then cooled for 8 min. The temperature change of the solution was monitored by a photothermal imager, and compared with the temperature change of a BPBBT solution dissolved in 5% THF at the same concentration (it is known that the photothermal conversion efficiency of BPBBT is 22% at this concentration), the photothermal conversion efficiency of BPBBT dot was calculated as 30.5%.

The method for calculating the photothermal conversion effect: the ratio of AT of both at the same time is multiplied by 22% (the photothermal conversion efficiency of BPBBT is known to be 22%).

BPBBT was mixed with DSPE-PEG-2000 to form BPBBT dots with bright NIR-II fluorescence with quantum yield (QY) and photothermal conversion efficiency as high as 3.47% and 30.5%, respectively. The fluorescence intensity and photothermal conversion effect of the BPBBT dot reach the maximum value at a mass ratio of 4:5, that is, the fluorescence intensity and photothermal conversion efficiency of the nanoparticle loaded with the NIR-II AIE dye prepared in Example 1 are the best.

### Effect Example 5. Test of In Vitro Photostability

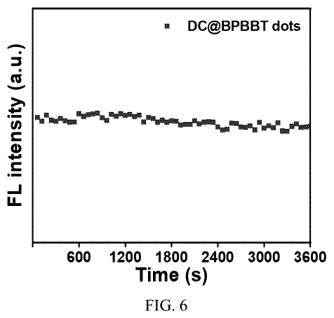

The nanoparticle loaded with the NIR-II AIE dye prepared in Example 1 was tested in vitro for fluorescence intensity at different time periods after 808 nm irradiation, and the fluorescence intensity was almost unchanged, indicating that the DC@BPBBT dot has good photostability. The process comprises the following steps: the DC@BPBBT dot solution was filled in a cuvette, continuous excitation is carried out for 1 hour by using an 808 nm laser, then a two-zone fluorescence spectrum was collected by using a fluorescence spectrometer, and the change of the fluorescence intensity of the material at 980 nm was analyzed, so that the good light stability of the material was judged. FIG. 6 shows the change of fluorescence intensity at 980 nm with time for DC/a, BPBBT dot solution.

### Effect Example 6. Test of In Vitro Photothermal Conversion Stability

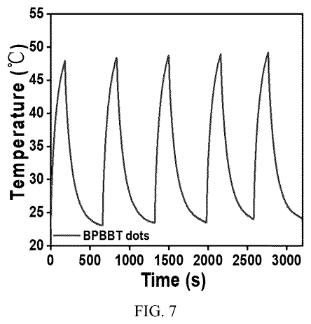

The BPBBT dot particles prepared in Example 1 were subjected to stabilization of the photothermal conversion effect during five heating-cooling cycles. The nanoparticle solution filled with the BPBBT dot was fixed and irradiated by using an 808 nm laser. Meanwhile, the temperature of the solution was monitored in real time by using a thermal imaging camera. The laser was turned off after 3 min of irradiation, and the laser was turned on again when the temperature was recovered to the initial temperature. In this way, 5 cycles were repeated, and whether the highest temperature of each cycle was the same or not was observed so as to judge the stability of photothermal conversion. The maximum temperature of BPBBT (about 48° C.) did not drop significantly after 5 irradiation cycles, indicating that BPBBT has good photothermal stability in vitro (FIG. 7).

As can be known from the above test, the nanoparticle loaded with the NIR-II AIE dye provided by the present invention have stronger fluorescence intensity, light stability and photoluminescence characteristics, can be applied to biological imaging of blood vessels, bile ducts, gastrointestinal tracts and the like, and have the characteristics of higher photothermal conversion efficiency and stable in vivo light conversion efficiency, so that the application of the nanoparticle in the medical field is expanded.

Photothermal therapy (PTT) is an emerging therapy for treating tumors by thermally ablating tumor cells with little side effects on normal organ systems. The nanoparticle loaded with the NIR-II AIE dye provided by the present invention has higher photothermal conversion efficiency and can be used for the treatment of tumors.

### Effect Example 7. Safety and Biocompatibility During Tumor Treatment

In vivo NIR-II fluorescence imaging: BALB/C mice were randomized into two groups when 4T1 tumor volume reached approximately 500 mm3, the tail vein was injected with BPBBT dot or DC@BPBBT dot (100 L, 200 g/mL). An NIR-II fluorescent small animal in vivo imaging system with a band-pass filter of 1000-1700 nm was adopted to acquire NIR-II images at 0, 3, 6, 12, 18, 24 and 48 h. The excitation wavelength was 808 nm. Mice were sacrificed 60 h after injection. Collecting main organs and tumors for fluorescence biodistribution analysis and imaging.

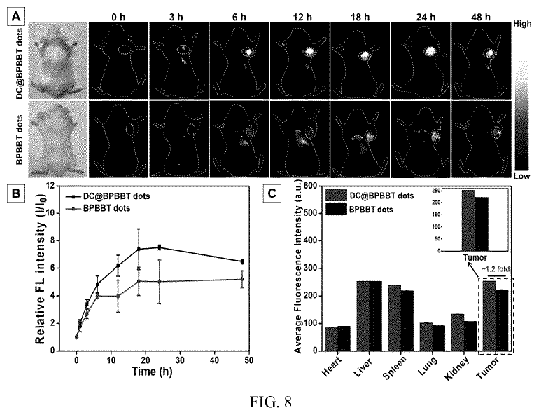

The results of the experiment are shown in FIG. 8, wherein A in FIG. 8 is the distribution of BPBBT dots versus DC@BPBBT dots in different organs over time. B is the time-dependent fluorescence intensity analysis of the tumor site. C is the distribution quantitative analysis of DC@BPBBT dot and BPBBT dot in different tissues. Error bar: mean±s.d (n=3).

It can be seen that there is a significant difference between DC@BPBBT dots and BPBBT dots in tumors, which are more enriched in tumor tissues.

### Effect Example 8. Evaluation of Cytotoxicity

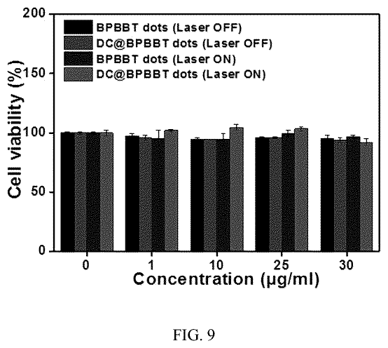

4T1 cells were inoculated in 96-well plates at a cell density of 5×10⁴ per well. After 24 h of culture at 37° C. in an environment containing 5% CO2, the culture was replaced with fresh media containing 0), 1, 10, 25, and 30 μg/mL of DC@BPBBT dot or BPBBT dot for 2 h, then the light group was irradiated for 15 min, while the dark group was not treated, and then cell viability was measured with CCK8-kit after further 24 h of culture in the incubator. The results of the experiment are shown in FIG. 9.

### Effect Example 9. Evaluation of Biocompatibility and Toxicity In Vivo

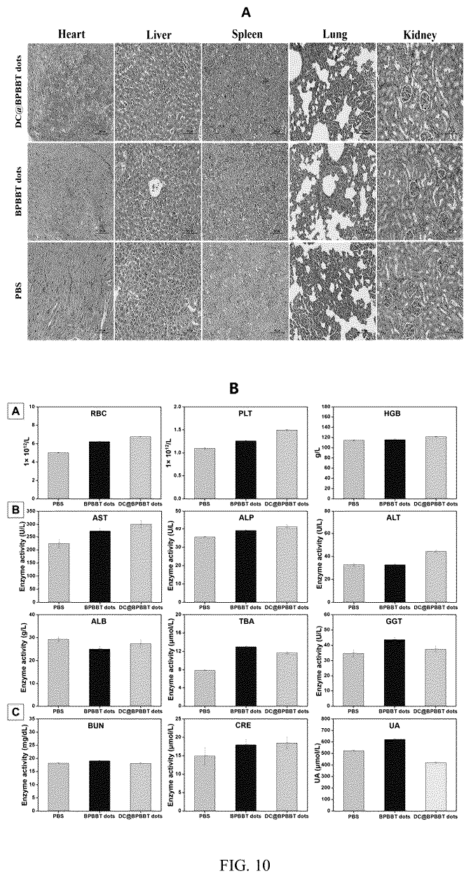

Mice were sacrificed 60 h after injection of PBS, BPBBT drops or DC@BPBBT drops, and major organs (heart, liver, spleen, lung, kidney) were taken. These organs were fixed in 10% neutral buffered formalin and then routinely processed into paraffin wax, with a slice thickness of 4 μm and stained with hematoxylin and eosin (H&E). The h&e stained sections were imaged by light microscopy and evaluated by three independent pathologists blinded to the project, and the experimental flow is shown in A of FIG. 10.

In addition, blood samples were collected and RBC, PLT, HGB, AST, ALP, ALT, ALB, TBA, GGT, BUN, CRE and UA counts were measured by Servicebio Corporation, Wuhan, China, and the statistics are shown in B of FIG. 10. Statistical analysis: all results were reported as mean±SD. The difference between groups adopts single-factor variance analysis and student t-test: *P<0.05, **P<0.01.

Compared with the PBS group, the parameters of the DC@BPBBT dot and BPBBT dot treatment group are normal, and the difference is not statistically significant (P>0.05).

These results confirm that the prepared DC@BPBBT dot has good in vivo safety and biocompatibility.

### Effect Example 10. Killing Efficiency of DC@BPBBT Dot Cells In Vitro and Mild PTT (about 42° C.)

Calcein protein (Calcein)/PI cell staining imaging method: it was found experimentally that PBS, BPBBT dot without T cells, and DC@BPBBT dot without T cells treated tumor cells showed a large amount of green fluorescence due to calcein-AM emission by living cells.

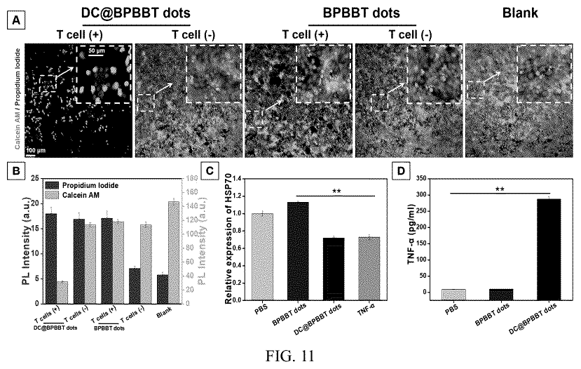

However, in the 808 nm laser irradiated and T cell treated DC@BPBBT dot group, green fluorescence was significantly reduced, and only a large amount of red fluorescence, i.e., propidium iodide (PI) stained dead cells, was observed. Thus, the results indicate that the DC@BPBBT dot-treated tumor cells are indeed more sensitive to heat than the control tumor cells, see A in FIG. 11.

In addition to the thermal susceptibility of tumor cells, the survival of immune cells during PTT was also of interest. Thus, the cell viability of CD3+ T cells was tested using the CCK-8 kit by treatment with 808 nm laser radiation at 42° C. for 15 min.

CCK-8 kit: with CCK-8 kit, irradiating the cells for 15 min by 808 nm laser at 42° C., and detecting the cell activity of the CD3+ T cells.

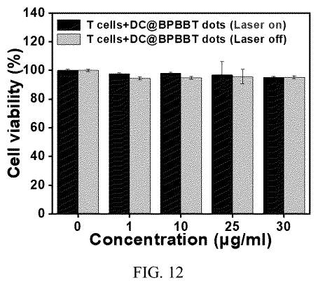

The experimental results show no significant decrease in T cell viability during PTT, indicating that damage to T cells by DC@BPBBT dots is negligible, and the results are shown in FIG. 12.

Next, Transwell Laboratory investigates whether the DC@BPBBT dot promotes T cells to secrete TNF-α and thereby has an effect of inhibiting the expression of heat shock protein families (including HSP27, HSP70 and HSP90) in tumor cells.

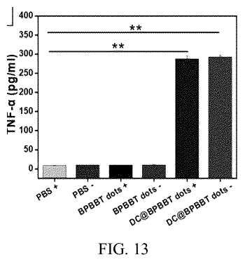

Inhibitory tumor microenvironment (TME) was simulated in vitro using a Transwell chamber. 4T1 breast cancer cells were inoculated in the upper chamber (pore size: about 0.4 μm) and CD3+ T cells were placed in the lower chamber. After 48 h of co-incubation of the two cell types, DC@BPBBT dot or BPBBT dot, PTT treatment was performed by irradiating the Transwell chamber with an 808 nm laser, and the temperature was maintained at about 42° C. for 15 min. In addition, after further incubation at 37° C. for 24 h, 4T1 cells and supernatant were collected for analysis in the following experiment. The level of TNF-α in the supernatant was then analyzed using an enzyme-linked immunosorbent assay (ELISA) method and a significant increase in the amount of TNF-α was observed after treatment with the DC/a, BPBBT dot (FIGS. 11D and 13).

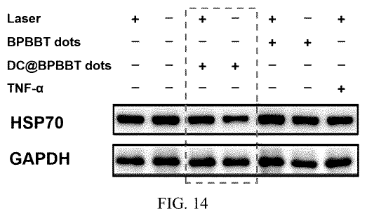

In addition, expression of HSP70 protein in 4T1 cancer cells was assessed using western blot after different treatments, wherein glyceraldehyde-3-phosphate dehydrogenase (GAPDH) was used as control (FIG. 14). Experiments show that HSP70 significantly reduces the relative expression of DC@BPBBT dots in 4T1 cancer cells (FIG. 11C). However, treatment with BPBBT dots has little effect on expression, indicating that the cell membrane protein component DC a, BPBBT dots play an important role in inhibiting the expression of HSP70.

Therefore, it is confirmed that the DC@BPBBT dot can activate T cells, and thus secretion of tumor necrosis factor (TNF-α) inhibits expression of HSP70 at tumor sites, increasing thermal sensitivity of tumor cells.

Photothermal therapy has factors that reduce the therapeutic effect during the treatment. Tumor cells are more sensitive to 42-45° C. than normal tissue cells, and compared with PTT at the hyperthermia temperature (higher than 50° C.), and PTT at the mild temperature (about 42° C.) is more prominent because the PTT has less damage to surrounding healthy tissues, so the PTT at the mild temperature (about 42° C.) is more suitable for the photothermal therapy. However, cell damage, such as apoptosis, caused by hyperthermia at mild temperatures (about 42° C.), can be repaired with the help of heat shock proteins (HSPs). The heat stimulus increases the expression of the tumor cell heat shock protein HSP70, making the tumor cell insensitive to heat, thereby limiting the effect of PTT. HSPs induce the development of therapeutic drug resistance, reducing the efficiency of therapy.

### Effect Example 11. In Vivo Anti-Tumor Therapy

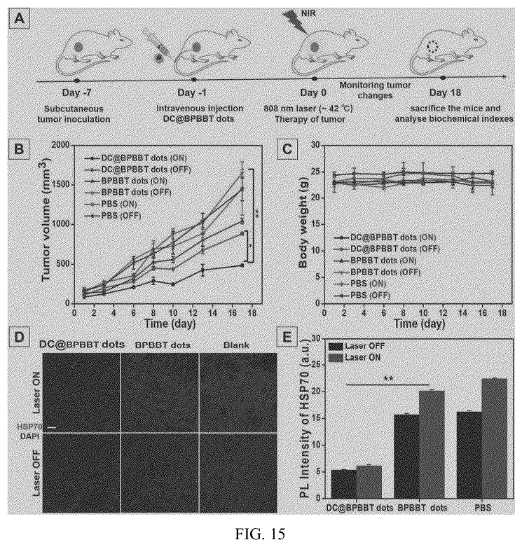

FIG. 15A shows the experimental procedure for photothermal efficacy of targeted tumors in mice. Each mouse was first inoculated subcutaneously with 4T1 tumor cells to establish an orthotopic xenograft breast cancer model.

Mice were randomly divided into two groups and each with 3 mice, 5×10⁶ 4T1 cells were injected subcutaneously near the upper limb on the right side of each mouse, 100 μL phosphate buffered saline (PBS), (1) BPBBT dot solution prepared in Example 1 and (2) DC@ABPBBT dot solution prepared were injected through the tail vein, respectively, when the tumor volume reached 50-70 mm3, and after 24 h, the tumor site was subjected to photothermal therapy with an 808 nm laser, and the temperature of the tumor site was monitored in real time with an infrared imaging camera and maintained for 5 min when the temperature reached 42° C. The size of the tumor and the weight of the mice were then recorded every two days to evaluate the photothermal therapy effect.

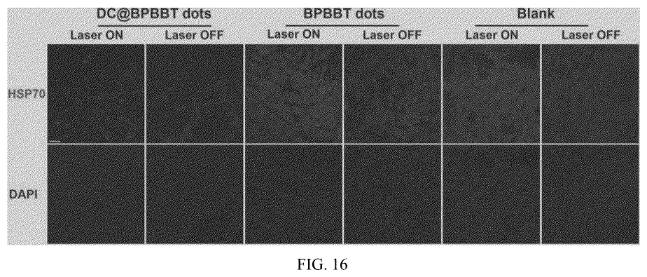

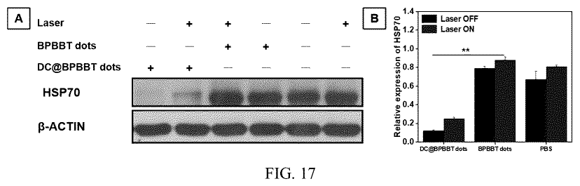

The body temperature of DC@BPBBT dot-treated mice increase more rapidly under 808 nm laser irradiation than did the PBS and BPBBT dot group over the same time period. These results further demonstrate the highly efficient tumor-targeting ability of the DC/a, BPBBT dot. We monitor the growth process of the tumor by measuring the tumor size every two days (FIG. 15B). Notably, tumor growth is significantly delayed in mice inoculated with the DC a, BPBBT dots after mild PTT (around 42° C.). The mild PTT combined with DC@BPBBT dot has the potential of inhibiting tumors. The body weight of the mice is measured every two days during the treatment. There is no significant change in the body weight of mice several days after the DC a, BPBBT dot and mild PTT (around 42° C.) (FIG. 15C). In addition, we isolate tumors from groups of mice after PTT and detect expression of hsp70 using immunofluorescence and western blot. As shown in FIGS. 15D, 15E, 16 and 17, HSP70 levels are significantly reduced in tumor cells after DC@BPBBT dot treatment.

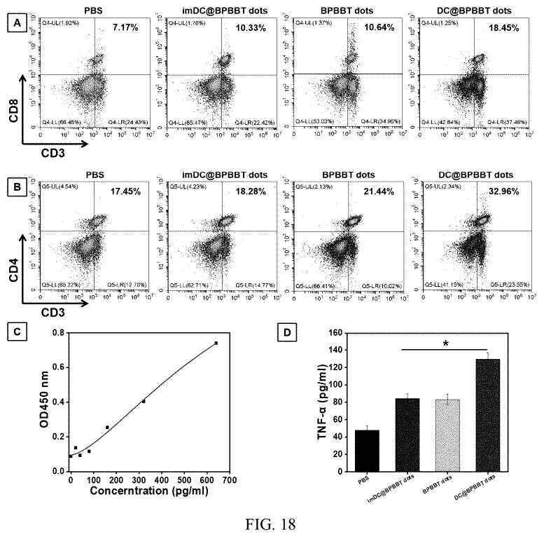

In addition, DC@BPBBT dots mediated immunotherapy is confirmed by analyzing the percentage of activated T cells and induced TNF-α expression of DC@BPBBT dots and in vivo BPBBT dots (FIG. 18). Tumor cells become more sensitive to heat and temperature due to decreased HSP70 levels and increased numbers of activated T cells and TNF-α levels in TME, and the photothermal effect of DC@BPBBT dots mediated immunotherapy combined with PTT (about 42° C.) inhibit tumor growth.

To further confirm that activation of T cells is initiated by membranes of DCs specifically activated by 4T1 cells, 4T1 tumor-bearing mice are randomly grouped and then injected with PBS, DC@BPBBT dots, imDC@BPBBT dots, and BPBBT dots, respectively, via the tail vein. At day 7, spleens and orbital bleeds of mice in each group were collected, and cells in the spleen were isolated and then analyzed by flow for T cell activation by incubating CD3, CD4, and CD8 fluorescent antibodies. For the orbital blood of mice, serum components were separated by centrifugation and then assayed for TNF-α content by ELISA kit. The results show that the percentage of CD3/CD8 and CD3/CD4 cells are significantly higher in the DC/a, BPBBT dots group than in the other groups (see FIG. 18), i.e., T cells are activated in large numbers; and the content of TNF-α in the serum of the DC@BPBBT dots is obviously higher than that of other groups, so that the immunotherapy is proved to be mediated by a mature DCs membrane, and the raised TNF-α can inhibit the expression of HSP70 at the tumor site, so that tumor cells are more sensitive to heat, and the two have a synergistic action, thereby inhibiting the growth of the tumor.

The present invention provides an intelligent nanoparticle-DC a, BPBBT dot coated by a DC membrane, wherein the BPBBT dot has the capability of NIR-II fluorescence imaging and photothermal conversion, and the outer DC membrane retains the capability of antigen presentation and T cell loading. Experimental results show that the enrichment efficiency of the BPBBT dot coated by the DC membrane at a tumor site is improved, and the activity of T cells in vivo is successfully activated, so that the tumor necrosis factor (TNF-α) is secreted to inhibit the expression of HSP70 at the tumor site, the thermal sensitivity of tumor cells is increased, and the growth of tumors is inhibited under low-temperature photothermal therapy. The present invention achieves the fluorescence imaging and delivery of the nanoparticle loaded with the NIR-II AIE dye through the DC biomimetic membrane, thereby achieving the diagnosis and treatment integration of the tumor.

The BPBBT dot in Example 1 can be arbitrarily replaced by a substance with NIR-II characteristic, AIE characteristic and photothermal effect, so that the adaptability of the NIR-II AIE dye with poor water solubility in fluorescence imaging can be improved, the thermal sensitivity of tumor cells can be increased, the growth of tumors is inhibited under low-temperature photothermal therapy, and the effect of integrating diagnosis and treatment of the tumors is achieved.

What is claimed is:

1. A DC biomimetic membrane nanoparticle loaded with an NIR-II AIE dye comprising a DC membrane and an NIR-II AIE dye loaded thereon, wherein the NIR-II AIE dye has photothermal characteristics.

2. The DC biomimetic membrane nanoparticle loaded with the NIR-II AIE dye according to claim 1, wherein the DC membrane and the NIR-II AIE dye loaded thereon are subjected to the following method to obtain the DC biomimetic membrane nanoparticle loaded with the NIR-II AIE dye:
dispersing DC cell membranes in PBS solution, then injecting the solution of the DC cell membranes through a filter by using a syringe, repeatedly operating so as to enable the cell membranes to be adhered to a filter membrane, aspirating the NIR-II AIE dye by using the syringe, enabling the NIR-II AIE dye to pass through the filter membrane having the cell membranes adsorbed thereto, and performing extrusion and filtration until the DC biomimetic membrane nanoparticle loaded with the NIR-II AIE dye is obtained.

3. The DC biomimetic membrane nanoparticle loaded with the NIR-II AIE dye according to claim 1, wherein the NIR-II AIE dye is a BPBBT dot.

4. The DC biomimetic membrane nanoparticle loaded with the NIR-II AIE dye according to claim 3, wherein the BPBBT dot is prepared by the following method:
dissolving DSPE-PEG-2000 and BPBBT in an organic solvent, dropwise adding the mixed solution into deionized water under an ultrasonic condition, continuously carrying out ultrasonic treatment until a uniform emulsion is formed, removing the organic solvent in the emulsion, and filtering the obtained emulsion by using a filter to obtain the BPBBT dot.

5. A method for preparing a DC biomimetic membrane nanoparticle loaded with an NIR-II AIE dye comprising the following steps:
S1: preparing the NIR-II AIE dye; and
S2: dispersing DC cell membranes in PBS solution, then injecting the obtained solution of the DC cell membranes through a filter by using a syringe to enable the cell membranes to be fully adhered to a filter membrane, aspirating the NIR-II AIE dye prepared in S1 by using the filter, enabling the NIR-II AIE dye to pass through the filter membrane having the cell membranes adsorbed thereto, and performing extrusion and filtration until the DC biomimetic membrane nanoparticle loaded with the NIR-II AIE dye is obtained.

6. The method for preparing the DC biomimetic membrane nanoparticle loaded with the NIR-II AIE dye according to claim 5, wherein the NIR-II AIE dye in S1 is a BPBBT dot and is prepared by the following method:
dissolving DSPE-PEG-2000 and BPBBT in an organic solvent, dropwise adding the mixed solution into deionized water under an ultrasonic condition, continuously carrying out ultrasonic treatment until a uniform emulsion is formed, removing the organic solvent in the emulsion, and filtering the obtained emulsion multiple times by using a filter to obtain the BPBBT dot.

7. The method for preparing the DC biomimetic membrane nanoparticle loaded with the NIR-II AIE dye according to claim 6, wherein a mass ratio of DSPE-PEG-2000 to BPBBT in S1 is 5:1 to 5:5, preferably 5:1, 5:2, 5:3, 5:4, or 5:5.

8. The method for preparing the DC biomimetic membrane nanoparticle loaded with the NIR-II AIE dye according to claim 6, wherein concentrations of DSPE-PEG-2000 and BPBBT in S1 in the organic solvent are 5 mg/mL and 1-5 mg/mL, respectively.

9. The method for preparing the DC biomimetic membrane nanoparticle loaded with the NIR-II AIE dye according to claim 6, wherein the ultrasonic treatment in S1 is carried out at a power of 100 W and a frequency of 40 KHz.

10. The method for preparing the DC biomimetic membrane nanoparticle loaded with the NIR-II AIE dye according to claim 5, wherein the DC cell membrane in S2 is obtained by a method comprising: isolating immature DCs from mouse bone marrow; stimulating with 4T1 cells and Poly I: C to obtain mature DCs, and then extracting to obtain DC cell membranes.

11. The method for preparing the DC biomimetic membrane nanoparticle loaded with the NIR-II AIE dye according to claim 5, wherein the filter membrane for the filter in S2 is a 0.22 μm filter membrane.

12. Use of the DC biomimetic membrane nanoparticle loaded with the NIR-II AIE dye according to claim 1 in the preparation of a reagent for in vivo imaging, a reagent for image-guided photothermal therapy and a reagent for drug delivery systems.

13. The use of the DC biomimetic membrane nanoparticle loaded with the NIR-II AIE dye in the preparation of the reagents for in vivo imaging, the reagents for image-guided photothermal therapy and the reagents for drug delivery systems according to claim 12, wherein the in vivo imaging or imaging guidance is in vivo imaging or imaging guidance in blood vessels, bile ducts and gastrointestinal tract in vivo.

14. Use of the DC biomimetic membrane nanoparticle loaded with the NIR-II AIE dye according to claim 1 in the preparation of a photothermal therapeutic drug or a photothermal therapeutic sensitizer for the treatment of a tumor.

15. Use of the DC biomimetic membrane nanoparticle loaded with the NIR-II AIE dye according to claim 1 in the preparation of a drug or reagent inhibiting expression of HSP70 at tumor sites, increasing thermal sensitivity of tumor cells and activating activity of T cells in vivo.

16. A method for inhibiting a tumor comprising administering to a subject the DC biomimetic membrane nanoparticle loaded with the NIR-II AIE dye according to claim 1.

17. The method for inhibiting the tumor according to claim 16 comprising performing low-temperature photothermal therapy.

18. The method for inhibiting the tumor according to claim 16, wherein the tumor comprises lung cancer, melanoma, breast cancer, prostate cancer, colon cancer, renal cell carcinoma, ovarian cancer, pancreatic cancer, hepatocellular carcinoma, and rhabdomyosarcoma.

19. A method for detecting a tumor comprising administering to a subject the DC biomimetic membrane nanoparticle loaded with the NIR-II AIE dye according to claim 1, and imaging the AIE dye therein.

20. A photothermal therapeutic drug or reagent comprising the DC biomimetic membrane nanoparticle loaded with the NIR-II AIE dye according to claim 1.
